(19) United States
(12) Patent Application Publication
(10) Pub. No.: US 2026/0355249 A1
(43) Pub. Date: Nov. 12, 2026

(54) METHOD FOR OBTAINING AN ADVANCED CELL THERAPY PRODUCT BY MEANS OF THE CULTURE OF MESENCHYMAL STEM CELLS CONDITIONED WITH HYALURONIC ACID AND SULFATED DERIVATIVES, FOR BONE INJURIES

(71) Applicants: CONSEJO NACIONAL DE INVESTIGACIONES CIENTÍFICAS Y TÉCNICAS (CONICET), Ciudad Autónoma de Buenos Aires (AR); UNIVERSIDAD NACIONAL DEL NOROESTE DE LA PROVINCIA DE BUENOS AIRES (UNNOBA), Junín Buenos Aires (AR)

(72) Inventors: Laura ALANIZ, Junín Buenos Aires (AR); Antonella ICARDI, Junín Buenos Aires (AR); Ina SEVIC, Junín Buenos Aires (AR); Daiana VITALE, Junín Buenos Aires (AR); Paolo ROSALES, Junín Buenos Aires (AR); Fiorella SPINELLI, Junín Buenos Aires (AR)

(73) Assignees: CONSEJO NACIONAL DE INVESTIGACIONES CIENTÍFICAS Y TÉCNICAS (CONICET), Ciudad Autónoma de Buenos Aires (AR); UNIVERSIDAD NACIONAL DEL NOROESTE DE LA PROVINCIA DE BUENOS AIRES (UNNOBA), Junín Buenos Aires (AR)

(21) Appl. No.: 19/488,679

(22) PCT Filed: May 29, 2024
(86) PCT No.: PCT/IB2024/055227

§ 371 Date: Nov. 28, 2025

(30) Foreign Application Priority Data
Jun. 1, 2023 (AR) P20230101398

(51) Int. Cl. A61K 35/28; A61P 19/08; C12N 5/0775

(52) U.S. Cl. CPC A61K 35/28; A61P 19/08; C12N 5/0665; C12N 2501/905; C12N 2506/1369

(57) ABSTRACT

The present description provides a method for promoting osteogenic differentiation and osteoinductive capacity of mesenchymal stem cells, comprising incubating said cells in the presence of hyaluronic acid or sulfated derivatives thereof. The described method enables the inoculation of allogeneic MSCs, expanded ex vivo, into a bone injury as a regenerative treatment of the injured bone tissue.

### TECHNICAL FIELD OF THE INVENTION

The present invention relates generally to the field of regenerative medicine, in particular to the field of bone tissue regeneration. More specifically, the invention refers to a method for obtaining an advanced cell therapy product which effectively induces bone regeneration in an injury.

### BACKGROUND OF THE INVENTION

The treatment of bone injuries which require the regeneration of bone tissue depends fundamentally on the presence of osteogenic and vasculogenic precursor cells in the surrounding tissues. The clinical standard for this kind of treatments is the use of autologous grafts due to perfect histocompatibility. However, the use of said autologous grafts exhibits significant disadvantages related to the limited amount of bone available to carry out the graft and the morbidity of the donor site. Another approach to bone grafting is allografts and xenografts, which solve the problem of limited autologous bone supply and do not require an additional surgical site for graft harvesting. However, allografts and xenografts have some drawbacks such as donor shortage, high costs, as well as the risk of transmission of infectious agents or immune reactions. Additionally, these strategies only allow repairing easily solved defects.

In recent years, mesenchymal stem cells (MSCs) in combination with biomaterials such as osteoconductive and osteoinductive scaffolds for these cells and growth factors have been used to accelerate bone healing at the site of injury (Mebarki et al., 2021). In this sense, MSCs play a key role in the bone healing process, not only by constituting osteoblast precursors, but also thanks to their capacity for differentiation, self-renewal and release of bioactive factors, and their immunomodulatory properties. Additionally, these cells have a natural tropism for injured zones. Therefore, supplementation with exogenous MSCs, in synergy with the migration and differentiation of these endogenous progenitor cells, could provide better results in the healing of critical-sized bone defects, i.e., those that do not repair spontaneously. Moreover, these precursor cells are known to migrate to the site of injury and differentiate into osteoblasts and endothelial cells, promoting bone formation and vascularization (Meijer et al. 2008), a critical process to regain full function. However, the aging process and certain pathologies affect the functionalities of these endogenous precursor cells, whereby the synergy with MSCs supplementation will depend on the physiological and pathological state of the individual to whom they are applied (laquinta et al., 2019). On the other hand, one of the main limitations associated with a cell therapy based on MSCs is that, given their great plasticity, more research is required to avoid problems related to unwanted differentiation of these cells once they enter the organism. In this regard, it is crucial to have standardized protocols to regulate MSCs growth conditions during ex vivo expansion (laquinta et al., 2019) thereof. In addition, although scaffolds are used to guide cell adhesion, proliferation and osteogenic differentiation of MSCs (Hench and Polak, 2002), it is still a challenge to create a scaffold that meets all the necessary properties for controlled and highly effective bone regeneration. These properties include osteoconduction, osteoinduction, biocompatibility, biodegradability, vascularization and retention of progenitor cells. In addition, an optimal porosity which allows a successful diffusion of nutrients, oxygen and cellular waste products must be achieved and, in general, it is very difficult to create the complex structure of the scaffolds with precise conventional techniques. On the other hand, exposure to osteoinductive growth factors is limited by the fact that it can induce osteogenesis in ectopic sites. Moreover, if a context of tumor bone injury is considered, proper targeting of MSCs differentiation is required to minimize the risks of malignant transformation.

Extracellular matrix factors such as hyaluronic acid can be combined with MSCs to modulate their behavior. Several documents (WO2015007797A1, US2011104230A1, AU2006200478B2, CN111494722A, CN111500533A, KR20110132579A, US2017354758A1, KR102017130B1) describe the use of hyaluronic acid to form a scaffold, matrix or hydrogel comprising MSCs in a three-dimensional array, which is then administered to the subject.

Patent application US2016184364A1, on the other hand, describes a method for promoting bone regeneration that involves administering MSCs to the patient in combination with hyaluronic acid, but separately.

However, there remains a need for more effective therapeutic strategies in the treatment of bone injuries.

### BRIEF DESCRIPTION OF THE INVENTION

The present invention provides a method for promoting osteogenic differentiation of mesenchymal stem cells (MSCs) comprising:
i—providing a standard culture medium suitable for the incubation of MSCs on a plate;
ii—seeding MSCs in the standard culture medium;
iii—incubating the MSCs in the standard culture medium to achieve their cell amplification;
iv—adding hyaluronic acid or a sulfated derivative thereof to the standard culture medium to generate a conditioned medium; and
v—incubating the MSCs in said conditioned medium.

In one embodiment of the invention, the sulfated derivative of hyaluronic acid has an average amount of sulfate groups per repeating unit of disaccharide within the range between 1 and 3. Preferably, the sulfated derivative of hyaluronic acid has an average amount of sulfate groups per repeating unit of disaccharide selected from the group consisting of 1 and 3.

In another embodiment of the invention, the hyaluronic acid or the sulfated derivative thereof is added to the standard culture medium in a concentration of between 10 μg/ml and 1000 μg/ml. Preferably, the hyaluronic acid or the sulfated derivative thereof is added to the standard culture medium in a concentration of 100 g/ml.

In another embodiment of the invention, the MSCs are incubated in the conditioned medium for 48 h.

In another embodiment of the invention, in step ii—of the method the MSCs are seeded in a plating density in the range between 5×10³ and 7.5×10³ cells/cm². Preferably, the MSCs are seeded in a plating density of 7.5×10³ cells/cm².

It is another aspect of the present invention to provide a composition for use in bone regenerative therapy comprising MSCs obtained from the method for promoting osteogenic differentiation of MSCs of the invention, suspended in a pharmaceutically acceptable carrier.

Another aspect of the present invention is to provide a method for the regenerative treatment of a bone injury in a subject, comprising:
i—providing a standard culture medium suitable for the incubation of MSCs on a plate;
ii—seeding MSCs in the standard culture medium;
iii—incubating the MSCs in the standard culture medium to achieve their cell amplification;
iv—adding hyaluronic acid or a sulfated derivative thereof to the standard culture medium to generate a conditioned medium;
v—incubating the MSCs in said conditioned medium; and
vi—inoculating the MSCs obtained after the incubation of step v—in the bone injury.

In one embodiment of this aspect of the invention, the sulfated derivative of hyaluronic acid has an average amount of sulfate groups per repeating unit of disaccharide within the range between 1 and 3. Preferably, the sulfated derivative of hyaluronic acid has an average amount of sulfate groups per repeating unit of disaccharide selected from the group consisting of 1 and 3.

In another embodiment of the invention, the hyaluronic acid or the sulfated derivative thereof is added to the standard culture medium in a concentration of between 10 μg/ml and 1000 μg/ml. Preferably, the hyaluronic acid or the sulfated derivative thereof is added to the standard culture medium in a concentration of 100 μg/ml.

In another embodiment of the invention, the MSCs are incubated in the conditioned medium for 48 h.

In another embodiment of the invention, the MSCs are seeded in a plating density in the range between 5×10³ and 7.5×10³ cells/cm². Preferably, the MSCs are seeded in a plating density of 7.5×10³ cells/cm².

In another embodiment of this aspect of the invention, the inoculation of the MSCs is carried out locally in the site of the bone injury.

### BRIEF DESCRIPTION OF THE FIGURES

FIG. 1. Chemical structures of hyaluronic acid and its sulfated derivatives wherein the average amount of sulfate groups per repeating unit of disaccharide is 1 or 3. A) Hyaluronic acid. B) Sulfated derivatives. In the HAs1 derivative, R₁, R₂ and R₃ are —H. In the HAs3 derivative, R₁, R₂ y R₃ are —SO₃Na/—H in a 2/1 ratio.

FIG. 2. Analysis of biological compatibility between HUCPVCs and HA and its sulfated derivatives. Viability analysis of the HA- and HAs-treated HUCPVCs at 24 h (A) and 48 h (B) from the start of the treatments by MTS. The results are relativized to the control and are represented as average±SEM, statistical analysis: two-way ANOVA and Bonferroni test. *p<0.05**p<0.01.

FIG. 3. Analysis of biological compatibility between HUCPVCs and HA and its sulfated derivatives. Comparison of cell viability between treatments at the same dose after 24 h (A) and 48 h (B) treatment. The results are represented as average±SEM, statistical analysis: two-way ANOVA and Bonferroni test.

FIG. 4. Analysis of biocompatibility between HUCPVCs and HA and its sulfated derivatives. Comparison of cell viability between both treatment times for HA (A), HAs1 (B) and HAs3 (C). The results are represented as average±SEM, statistical analysis: two-way ANOVA and Bonferroni test.

FIG. 5. Analysis of biological compatibility between HUCPVCs and HA and its sulfated derivatives. Percentage of early and late apoptotic cells. The results are represented as the average of a single experiment. A) HA-treated HUCPVCs. B) HAs1-treated HUCPVCs. C) HAs3-treated HUCPVCs.

FIG. 6. Evaluation of basal immunogenic capability of HUCPVCs. Quantification of the percentage of proliferative lymphocytes in absence (HUCPVCs−) and in presence (HUCPVCs+) of allogeneic stimulating HUCPVCs. The results are represented as average ±SEM, statistical analysis: T-test.

FIG. 7. Evaluation of immunogenic capability of HUCPVCs treated with HA and its sulfated derivatives by flow cytometry. Quantification of the percentage of proliferative lymphocytes in the presence of A) HA-treated HUCPVCs, B) HAs1-treated HUCPVCs, C) HAs3-treated HUCPVCs. The results are relativized to the base proliferation control (C−) and are represented as average±SEM, statistical analysis: one-way ANOVA and Tukey test. C+: proliferation by allogeneic activation control.

FIG. 8. Analysis of differentiation capability of HUCPVCs. Osteogenic differentiation with Alizarin Red S staining for calcification zones of HA-, HAs1- and HAs3-treated HUCPVCs. Relative frequence distribution analysis of integrated density of calcification zones. A) HA-treated HUCPVCs. B) HAs1-treated HUCPVCs. C) HAs3-treated HUCPVCs. Results are expressed in RawIntDen units.

FIG. 9. Analysis of differentiation capability of HUCPVCs. Osteogenic differentiation with Alizarin Red S staining for calcification zones of HA-, HAs1- and HAs3-treated HUCPVCs. Total integrated density. Values are expressed as arithmetic mean±SEM of the integrated density (in RawIntDen units) of the 8 zones evaluated per treatment, statistical analysis: one-way ANOVA and Tukey test. The images obtained once the staining was made (below) show the effect of the increasing concentrations of HA and HAs on the osteogenesis (10×). A) HA-treated HUCPVCs. B) HAs1-treated HUCPVCs. C) HAs3-treated HUCPVCs.

FIG. 10. Analysis of differentiation capability of HUCPVCs. Comparison of integrated density of calcification zones for the treatments evaluated at each different work concentration (0, 10, 100 and 500 μg/ml). Values are expressed as arithmetic mean±SEM of the integrated density of the 8 zones evaluated for each treatment, statistical analysis: two-way ANOVA and Bonferroni test. *p<0.05

FIG. 11. Analysis of differentiation capability of HUCPVCs. Adipogenic differentiation with Oil Red O staining for HUCPVC lipid vacuoles. Relative frequence distribution analysis of integrated density of the lipid vacuoles. A) HA-treated HUCPVCs. B) HAs1-treated HUCPVCs. C) HAs3-treated HUCPVCs. Results are expressed in RawIntDen units.

FIG. 12. Analysis of differentiation capability of HUCPVCs. Adipogenic differentiation with Oil Red O staining for HUCPVC lipid vacuoles. Total integrated density. Values are expressed as arithmetic mean±SEM of the integrated density (in RawIntDen units) of the 8 zones evaluated per treatment, statistical analysis: one-way ANOVA and Tukey test. *p<0.05. The images obtained once the staining was made show the effect of the increasing concentrations of HA and HAs on the adipogenesis (10×). A) HA-treated HUCPVCs. B) HAs1-treated HUCPVCs. C) HAs3-treated HUCPVCs.

FIG. 13. Analysis of differentiation capability of HUCPVCs. Comparison of integrated density of lipid vacuoles for the treatments evaluated at each different work concentration. Values are expressed as arithmetic mean±SEM of the integrated density of the 8 zones evaluated for each treatment, statistical analysis: two-way ANOVA and Bonferroni test.

FIG. 14. Results obtained in the evaluation of the potential of HUCPVCs treated with HAs1 and HAs3 to stimulate bone formation in a mechanical bone injury model, 28 days after surgery. A) Control group treated with HUCPVCs. B) Group treated with HUCPVCs− HAs1. C) Group treated with HUCPVCs− HAs3.

The dotted black lines (----) limit the zone of the defect. *: vacant defects, with absence of osteoid tissue (non-mineralized organic matrix). Inf: inflammatory cells. Mo: mononuclear immune cells (lymphocytes and monocytes). OS: osteoid tissue. Ob: osteoblasts. Oc: osteocytes. V: blood vessels. CH: Havers channels. OC: osteoclasts.

FIG. 15. Analysis of antitumor in vitro capability of the supernatants (sb) derived from HUCPVCs treated with HAs1 and HAs3. The obtained sb affect the growth properties of tumor cell lines derived from osteosarcoma (OS), SAOS-2.

### DETAILED DESCRIPTION OF THE INVENTION

Mesenchymal stem cells (MSCs) present features of multipotent stem cells, which have the capability to differentiate into cell types of mesodermal origin, such as adipocytes, osteocytes and chondrocytes, maintaining a high capacity for self-renewal. The present invention is based on the surprising finding by the inventors that incubation of MSCs in the presence of hyaluronic acid or sulfated derivatives thereof promotes osteogenic differentiation of MSCs to the detriment of adipogenic differentiation thereof.

Correspondingly, it is an aspect of this invention to provide a method for promoting osteogenic differentiation of MSCs comprising:
i—providing a standard culture medium suitable for the incubation of MSCs on a plate;
ii—seeding MSCs in the standard culture medium;
iii—incubating the MSCs in the standard culture medium to achieve their cell amplification;
iv—adding hyaluronic acid or a sulfated derivative thereof to the standard culture medium to generate a conditioned medium; and
v—incubating the MSCs in said conditioned medium.

MSCs can be isolated from a number of sources, such as bone marrow, adipose tissue and peripheral blood, as well as derived from perinatal sources, such as umbilical cord blood and tissue, placenta and amniotic fluid, and from lung or heart. The MSCs used in the methods of the present invention are preferably obtained from human umbilical cord tissue (hUC-MSCs), as one skilled in the art will appreciate. More preferably, the hUC-MSCs are cells obtained from the perivascular region of the human umbilical cord (HUCPVCs).

By “standard culture medium” it is to be understood any medium which allows to culture MSCs for its correct cell amplification. It is within the expected knowledge for a person of skill in the art to select an appropriate culture medium to this end. An example of a standard culture medium which may be used in the method of the invention is Dulbecco's modified Eagle's medium (DMEM). Preferably, the DMEM used has a low Glucose concentration (1 g/l).

The standard culture medium can also be supplemented with some additional ingredient to ensure proper incubation of MSCs in steps i—to v—, as a person of skill in the art will appreciate. For example, the standard culture medium can be supplemented with a nutrient ingredient such as fetal bovine serum (FBS), supplements usually used in the art such as L-glutamine, and antibiotics such as penicillin and streptomycin. In a particularly preferred embodiment, the standard culture medium provided in step i—of the method of the invention is DMEM of low Glucose concentration (1 g/l), supplemented with 2 μmol/L of L-Glutamine, 100 U/mL penicillin, 100 mg/mL streptomycin and 20% FBS.

Seeding of MSCs in the standard culture medium can be performed according to common practice in the state of the art, as anyone with average knowledge in the field will appreciate. In an embodiment of the invention, the MSCs are seeded in a plating density in the range between 5×10³ and 7.5×10³ cells/cm². Preferably, the MSCs are seeded in a plating density of 7.5×10³ cells/cm². The aforementioned plating densities are proposed assuming that total viable cells are used.

Step iii—of incubation of the MSCs in the standard culture medium should be carried out under conditions that ensure proper cell amplification of the seeded MSCs. Incubation conditions may be optimized by a person of skill in the art by resorting to their general knowledge, and carrying out routine experimentation.

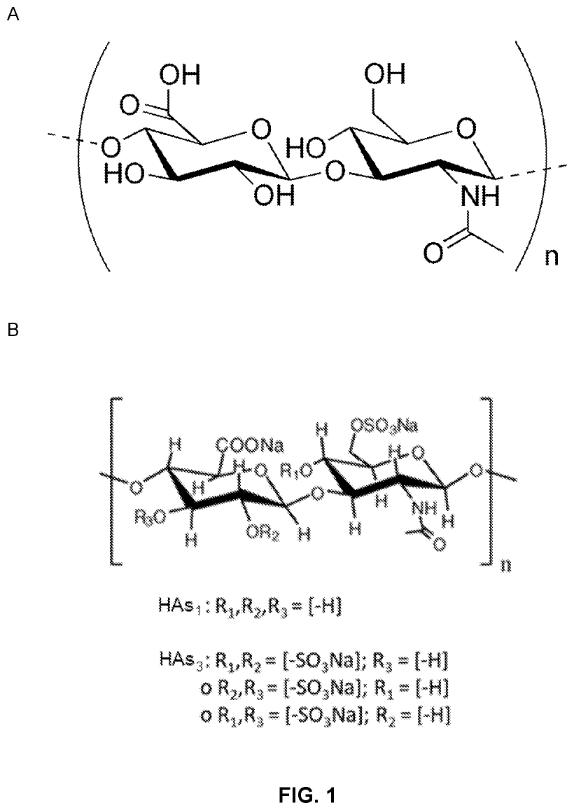

Hyaluronic acid is well known in the state of the art, and consists of a glycosaminoglycan formed by repeating disaccharide units constituted by D-glucuronic acid and N-acetyl-D-glucosamine. The term “sulfated derivative of hyaluronic acid” refers to hyaluronic acid to which sulfate groups have been incorporated along its chain by synthetic processes known in the state of the art. The sulfation degree of the hyaluronic acid chain may vary according to the synthesis conditions. FIG. 1 shows the structural differences between hyaluronic acid and its sulfated derivatives wherein the average amount of sulfate groups per repeating unit of disaccharide is 1 or 3. Sulfation of the hyaluronic acid chain offers the advantage of providing greater resistance to physiological degradation with respect to non-sulfated hyaluronic acid, and therefore greater bioavailability. Therefore, in a preferred embodiment, in step iv—of the method of the invention a sulfated derivative of hyaluronic acid is added to the standard culture medium. Preferably, the sulfated derivative of hyaluronic acid has an average amount of sulfate groups per repeating unit of disaccharide within the range between 1 and 3. More preferably, the sulfated derivative of hyaluronic acid has an average amount of sulfate groups per repeating unit of disaccharide selected from the group consisting of 1 and 3. Most preferably, the sulfated derivative of hyaluronic acid has an average amount of 3 sulfate groups per repeating unit of disaccharide.

The person skilled in the art will appreciate that hyaluronic acid or the sulfated derivative thereof, because of its polymeric character, can have a molecular weight that is comprised in a broad range. However, it should be noted that the molecular weight of these molecules is a relevant factor since the biological function of hyaluronic acid depends on its molecular weight. In a preferred embodiment, the hyaluronic acid or sulfated derivative thereof has a molecular weight in the range between 10 and 250 kDa. In a particularly preferred embodiment, in step iv—of the method of the invention a sulfated derivative of hyaluronic acid with a molecular weight within the range between 25 and 70 kDa is added.

Hyaluronic acid or the sulfated derivative thereof must be added to the standard culture medium thus generating a conditioned medium, and it is added at a final concentration such that it achieves the effect of promoting osteogenic differentiation of MSCs, without compromising their viability. In an embodiment of the invention, the hyaluronic acid or the sulfated derivative thereof is added to the standard culture medium in a concentration of between 10 μg/ml and 1000 μg/ml with respect to the total volume of the standard culture medium to generate the conditioned medium. For instance, the final concentration of the hyaluronic acid or the sulfated derivative thereof in the conditioned medium thus obtained may be 10 μg/ml, 100 μg/ml, 500 μg/ml, or 1000 μg/ml. Preferably, the hyaluronic acid or the sulfated derivative thereof is added to the standard culture medium in a final concentration of 100 μg/ml, thus generating the conditioned medium.

Incubation of the MSCs in the conditioned medium comprising the hyaluronic acid or the sulfated derivative thereof should be carried out under conditions such that MSC viability is ensured and adequate promotion of osteogenic differentiation of MSCs is achieved. It is within the general knowledge expected for a person of skill in the art to optimize the conditions under which the incubation of MSCs is carried out in step v—of the method, depending on the resources and experimental arrangements available. Of particular relevance is the duration of the incubation, as it should be sufficient to generate an adequate degree of promotion of osteogenic differentiation of the MSCs. In particular, in step v—of the method preferably MSCs are incubated for at least 24 h, more preferably for 48 h.

The method for promoting osteogenic differentiation of MSCs of the present invention has as a product MSCs induced toward osteogenic differentiation. Said MSCs are ready to be inoculated into a subject suffering from a bone injury that requires bone tissue regeneration, after which, thanks to their capacity to secrete bioactive factors and various signals, they will generate a local microenvironment that will stimulate the tissue regeneration process, in addition to the fact that, under the stimulus with hyaluronic acid or its sulfated derivative, these cells will have a restricted cellular fate. The present invention has as an advantage, therefore, that it is not necessary to administer the MSCs together with scaffolds or other three-dimensional structures, as proposed in the state of the art, which can be of complex construction. The present invention, on the other hand, is based on “educating” MSCs exogenously, prior to their use, compromising their phenotypic and functional characteristics, so as to guarantee the desired therapeutic effect by means of a method of simple implementation.

For them to be administered to a subject suffering from a bone injury, the MSCs induced towards osteogenic differentiation obtained by the method of the present invention must be suspended in a carrier suitable for inoculation into the subject's injury by injection.

Correspondingly, it is another aspect of the present invention to provide a composition for use in bone regenerative therapy, comprising MSCs induced towards osteogenic differentiation obtained according to the method of the invention, suspended in a pharmaceutically acceptable carrier. Said carrier must be a medium that maintains the viability of the MSCs during storage of the composition, while it must be compatible for administration to a subject who has suffered a bone injury to be treated. Several carriers which may be suitable to this end are known, as a person of skill in the art will appreciate. In a particular embodiment of this aspect of the invention, the carrier is DMEM medium supplemented with L-glutamine.

In a preferred embodiment, the composition comprises MSCs induced towards osteogenic differentiation by their incubation with a sulfated derivative of hyaluronic acid which has an average amount of sulfate groups per repeating unit of disaccharide within the range between 1 and 3. More preferably, the sulfated derivative of hyaluronic acid has an average amount of sulfate groups per repeating unit of disaccharide selected from the group consisting of 1 and 3. Most preferably, the sulfated derivative of hyaluronic acid has an average amount of 3 sulfate groups per repeating unit of disaccharide.

In another preferred embodiment, the MSCs comprised within the composition are hUC-MSCs. Even more preferably, the hUC-MSCs are HUCPVCs.

The amount of MSCs required for their administration to a subject will depend on the size (area in cm²) of the bone injury to be treated. Therefore, the concentration of the MSCs in the composition according to the invention will be customized, adjusted to the requirements of the patient and destined to a single use.

The purpose of the composition of this aspect of the invention is to provide a suitable vehicle for the inoculation of the MSCs obtained by the method of promoting osteogenic differentiation of the present invention. Correspondingly, in an embodiment of this aspect of the present invention, the aforementioned composition is for use in the regenerative treatment of a bone injury.

Considering the therapeutic effect of the MSCs induced towards osteogenic differentiation obtained by the method of the present invention, it is another aspect of the invention to provide a method for the regenerative treatment of a bone injury comprising obtaining MSCs induced towards osteogenic differentiation, and inoculating said MSCs into the bone injury. Correspondingly, this aspect of the invention provides a method for the regenerative treatment of a bone injury in a subject, comprising:
i—providing a standard culture medium suitable for the incubation of MSCs on a plate;
ii—seeding MSCs in the standard culture medium;
iii—incubating the MSCs in the standard culture medium to achieve their cell amplification;
iv—adding hyaluronic acid or a sulfated derivative thereof to the standard culture medium to generate a conditioned medium;
v—incubating the MSCs in said conditioned medium; and vi—inoculating the MSCs obtained after the incubation of step v—in the bone injury.

All of the considerations raised above for the method for promoting osteogenic differentiation of MSCs of the invention apply also to the method for regenerative treatment of a bone injury in a subject in accordance with this aspect of the invention.

The bone injuries to be treated by means of the method of this aspect of the invention are those which exceed the critical size (>2 cm) or which are hard to heal. For example, the injury to be treated may be selected from the group consisting of bone injuries present in traumatic injuries, degenerative diseases (osteoporosis and osteopenia), congenital defects or surgical excision associated with tumors.

The MSCs obtained by the method of the invention further exhibit antitumor activity, which makes the method for the regenerative treatment of a bone injury described herein particularly useful for the treatment of a bone injury generated by a tumor. Correspondingly, in a particular embodiment of this aspect of the invention, the bone injury to be treated is caused by a tumor.

The inoculation of the MSCs to the subject's bone injury is carried out by techniques known to the person with average skill in the technical field. In particular, the MSCs are implanted locally by means of an injection into the site of the injury, which may be carried out by a medical specialist. The required dose will depend on the size of the bone injury to be treated. The medical professional will know how to adjust the treatment to the needs of each patient, also taking into account the characteristics of the patient in question, such as age, co-morbidities, etc.

### Examples

### Cell Culture and General Information

Umbilical cord MSCs (hUC-MSCs) grow in surface adherence. For the initial stage of cell amplification, cultures of hUC-MSCs obtained from the perivascular region of the human umbilical cord (HUCPVCs) were established in monolayer seeding at a plating density of 2000 cells/cm², in sterile Petri dishes of 140 cm² diameter. Cells were cultured in DMEM medium (Lab. Microvet) with low glucose concentration, supplemented with 2 μmol/L glutamine (Lab. Microvet), 100 U/mL penicillin (Lab. Microvet), 100 mg/mL streptomycin (Lab. Microvet) and 20% fetal bovine serum (FBS, Natocor). This medium will be referred to as “standard culture medium” throughout the descriptions of the Examples below.

HUCPVCs were kept in an oven at 37 °C and in a humid atmosphere of 5% CO₂. For each amplification and passage the cells were removed with Trypsin and plated at optimal density and used specifically at passage 7 from isolation.

HUCPVCs were cultured on corresponding plates according to the requirements of the subsequent experiment and maintained with their standard culture medium, with 5% FBS, for 24 h prior to the treatments for their stabilization and necessary adherence.

The hyaluronic acid (HA) used in the following Examples was obtained from Fidia Farmaceutici S.p.A. (Abano Terme, PD, Italy). The sulfated derivatives were chemically synthesized from the tetrabutylammonium salt of HA (Hintze et al. 2009, Pavan et al. 2016). Briefly, for the synthesis of HAs1 and 3 (mean number of sulfate groups per repetition of disaccharide unit [DS] was 1 and 3, respectively), a SO₃-pyridine complex (polymer/SO₃ molar ratio was 1:4 and 1:10, respectively) was used as sulfation agent in DMSO (20 h, room temperature). The sulfated products were precipitated in ethanol and purified by dialysis 72 h in distilled water using a membrane with a 12 kDa cutoff to eliminate the salts. Then, they were freeze-dried. The sulfation degree was determined by measurements of sulfur content by ICP-OES (Perkin Elmer) at 181.975 nm (wavelength emission of sulfur) (Table 1).

TABLE 1
Information of the HAs used

| | Molecular | | | Sulfation |
|---|---|---|---|---|
| HAs type | weight (kDa) | Recovery % | Total (g) | degree |
| HAs1 | ≈28.1 | 53.7 | 2.58 | 0.74 |
| HAs3 | ≈66.4 | 83.7 | 4.19 | 2.53 |

Two sulfated derivatives were used: derivative with one mean sulfate group per repeated disaccharide unit (HAs1), and derivative with three mean sulfate groups per repeated disaccharide unit (HAs3). The molecular weight of HAs1 was 28.1 kDa, and the one of HAs3 was 66.4 kDa.

Student's test (T-test) was used to make comparisons between two experimental groups, and one-way analysis of variance (ANOVA) followed by Tukey's test to compare the means of more than two experimental groups. Two-way ANOVA, followed by the Bonferroni test, was also used for multiple comparisons using GraphPad Prism 5.0, considering a p<0.05 as statistically significant.

### Example 1-Analysis of Biological Compatibility Between HUCPVCs and Hyaluronic Acid (HA) and its Sulfated Derivatives (HAs)

### MTS Assays

To analyze the biological compatibility between HUCPVCs and HA and HAs, and to determine the time and dose of use that maintain their viability, cell viability assays were performed after treatment with these molecules using the MTS technique. This is a sensitive colorimetric method for the quantification of viable cells, which is based on the reduction of an MTS tetrazolium compound by viable cells to generate a colored formazan dye that is soluble in cell culture media. This conversion is carried out by NADPH-dependent dehydrogenase enzymes in metabolically active cells and the formazan dye produced is quantified by measuring absorbance at 490 nm and provides a measure of cell viability. To approach this assay, HUCPVCs were seeded in a 96-well plate, maintaining a plating density of 7.5×10³ cells/cm², with the addition of 100 μl of standard culture medium supplemented with 5% FBS, and maintained for 24 h prior to treatment with the conditioned medium with HAs and HAs, for their stabilization and necessary adherence. Upon reaching 80% confluence, the HUCPVCs were treated with the conditioned medium, at the final HA and HAs concentrations of 0.1; 10; 100; 500 and 1000 μg/ml, in addition to including the necessary controls for the assay. The viability analysis was performed after 24 and 48 h of cell culture in the presence of the conditioned medium. For this purpose, 7 μL of MTS Reagent (ab197010; Abcam, Cambridge, UK) was added to each well and the cells were incubated in the dark for 3 h at 37 °C and 5% CO₂ in an oven. The absorbance in each well was measured at a wavelength of 490 nm using a spectrophotometric plate reader, taking measurements every 45 minutes.

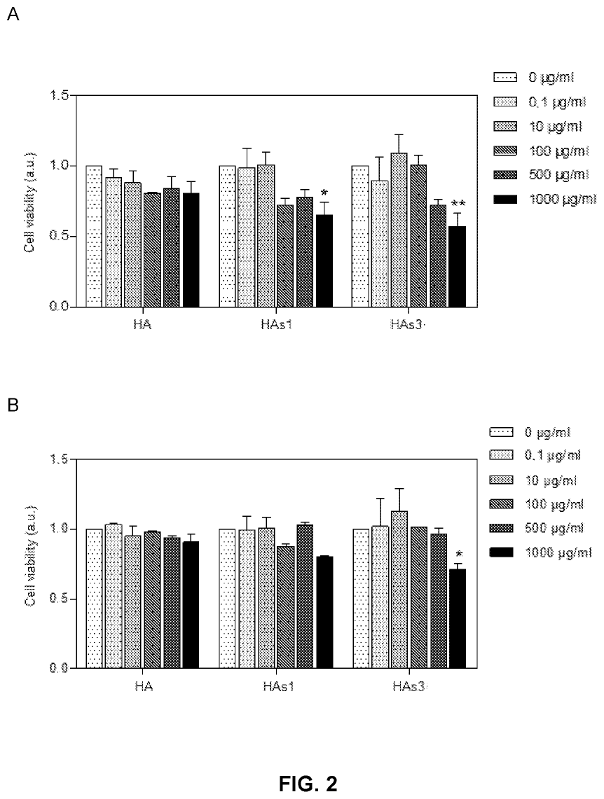

When the cell viability of HUCPVCs treated with HA and HAs was analyzed with respect to the untreated control (CB), it was determined that HAs1 (0.1 μg/ml=0.9850±0.1420; 10 μg/ml=1.011±0.0880; 100 μg/ml=0.7255±0.04750; 500 μg/ml=0.7765±0.05450; 1000 μg/ml=0.6535±0.08750) and HAs3 (0.1 μg/ml=0.8955±0.1685; 10 μg/ml=1.090±0.1315; 100 μg/ml=1.011±0.06950; 500 μg/ml=0.7255±0.03750; 1000 μg/ml=0.5690±0.0970) significantly reduced cell viability at the highest concentration used (1000 μg/ml), after 24 h of treatment (FIG. 2A). However, after 48 h, significant differences were only observed for HAs3 treatment (0.1 μg/ml=1.020±0.2000; 10 μg/ml=1.127±0.1625; 100 μg/ml=1.013±0.001500; 500 μg/ml=0.9675±0.04050; 1000 μg/ml=0.7095±0.04450) for this same concentration (FIG. 2 B). In this regard, viability was found to be dependent on the concentration of HA and HAs used in the assays, as viability was observed to decrease as the concentration used increased.

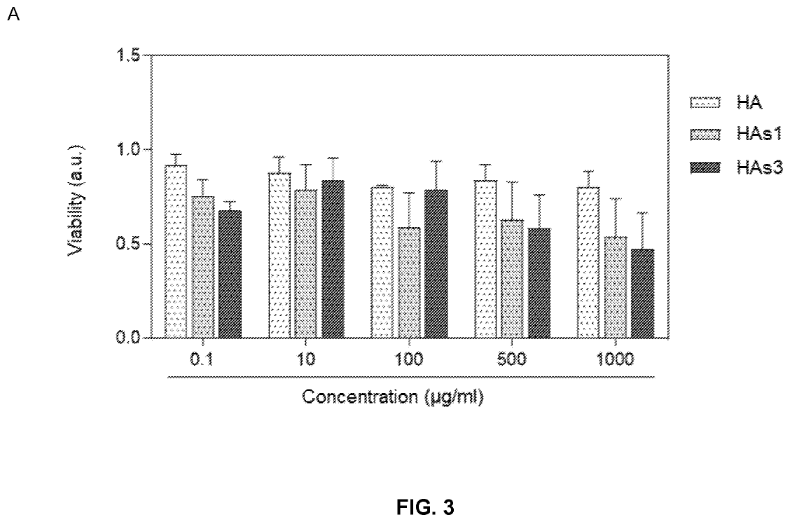

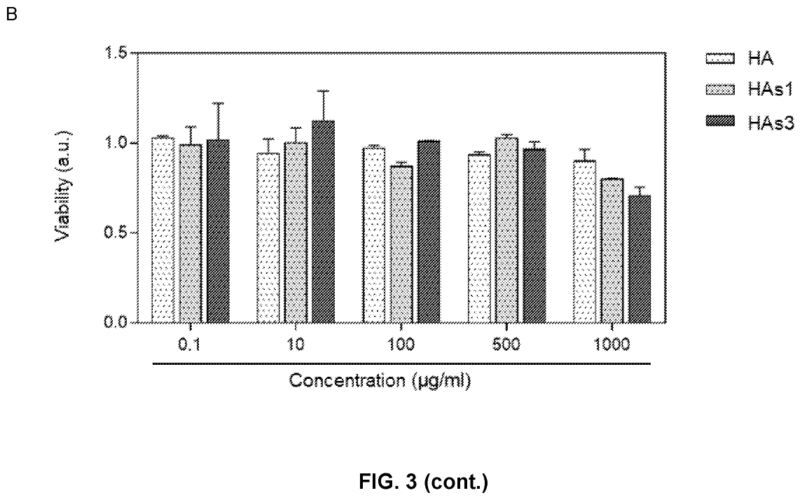

Moreover, viability turned out not to be directly dependent on the compound used, as no significant differences in the viability of HUCPVCs were detected for the different HAs at the same dose, at any of the times evaluated (FIGS. 3A and B).

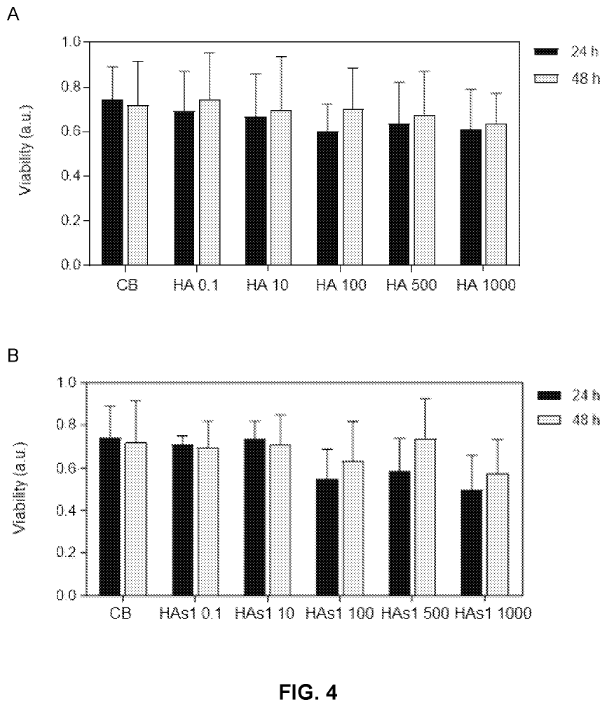

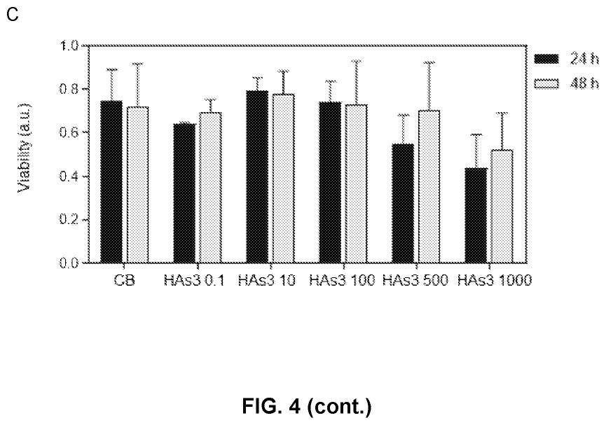

On the other hand, there was a marked trend of increased cell viability at 48 h of treatment compared to 24 h, although the differences between the two times evaluated were not statistically significant (FIG. 4). This result indicates that 48 h is the optimal time condition to obtain viable cells cultured in the conditioned medium.

### Flow Cytometry

To analyze the biological compatibility between HUCPVCs and HA and HAs, assays for apoptosis with AnnexinV-APC (ImmunoTools) and necrosis with Propidium Iodide (PI) were also performed by flow cytometry. Apoptosis and necrosis are the two main forms of cell death. Apoptotic cells change the structure of their membrane exposing the phosphatidylserine (PS) in its surface. This redistribution of PS from the inner surface to the outer cell membrane and its detection by interaction with AnnexinV represents an early indicator of apoptosis. Whereas, in early stage apoptosis, plasma membrane integrity excludes PI in viable and apoptotic cells, such that cells stain with AnnexinV, but not with PI. In late stage apoptosis, when the cell membrane loses its integrity, necrotic cells become permeable to PI. This allows performing a dual-parameter FACS analysis that allows discrimination between viable, apoptotic and necrotic cells, allowing the recording of non-apoptotic cells (annexin V-APC negative/PI negative), cells in early apoptosis (annexin V-APC positive/PI negative) and necrotic cells (annexin V-APC positive/PI positive). To perform this assay, 2.5×10⁵ cells were treated with HA and HAs, at the concentrations of 10, 100 and 1000 μg/ml, for 24 and 48 h. Upon completion of the treatment period, the supernatant from each well was pooled, centrifuged at 2100 rpm for 10 min, and the supernatant obtained was discarded, in order to collect cells in suspension if present and indicative of undergoing a death process. Also, 800 μL of cold EDTA (1 mM) was added per well and allowed to act for 5 minutes at 4 °C. Cells from each well were pooled in the same vial from which the corresponding supernatant was discarded. Centrifugation was carried out again at 2100 rpm for 10 minutes and the supernatant was discarded, followed by 2 washes with 500 μL of cold PBS 1X at 2100 rpm for 10 minutes. The resulting pellet was resuspended in 200 μL of FACS buffer, 5 μl of AnnexinV-APC was added and it was incubated for 30 minutes at room temperature in the dark. It was washed with 200 μL of Binding Buffer 1×, and finally resuspended in 200 μL of FACS Buffer, and analyzed by flow cytometry. PI was added at the time of passing the cells through the cytometer; 1 μL of PI was added to each sample. The assay included the necessary controls; an autofluorescence control and compensation controls, a positive AnnexinV-APC control, a positive PI control, and a positive control for both. Positive apoptosis controls were obtained by incubating cells with 3% formaldehyde (in PBS) for 30 minutes. The entire technique was performed on ice, and the steps of centrifugation at 4 °C and addition of the reagents were carried out in darkness. Sample analysis was performed on a FACS Canto II flow cytometer (BD BioSciences) and the data obtained were analyzed using the software FlowJo, LLC.

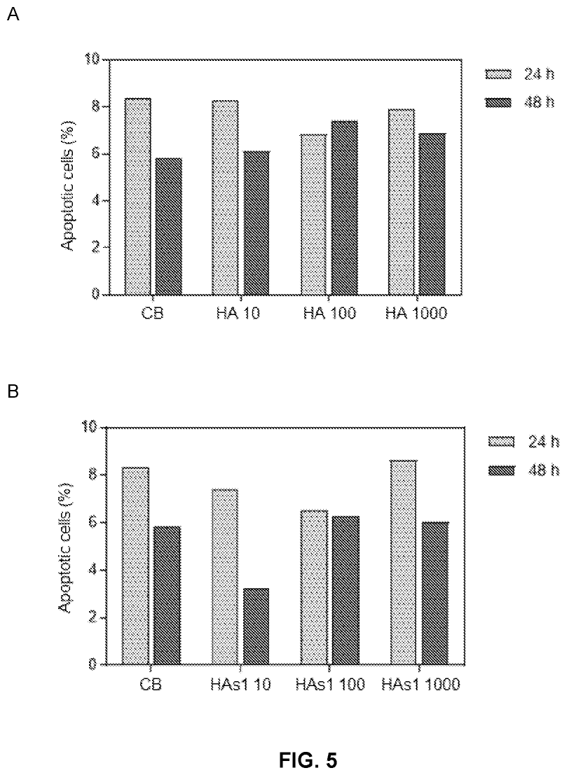

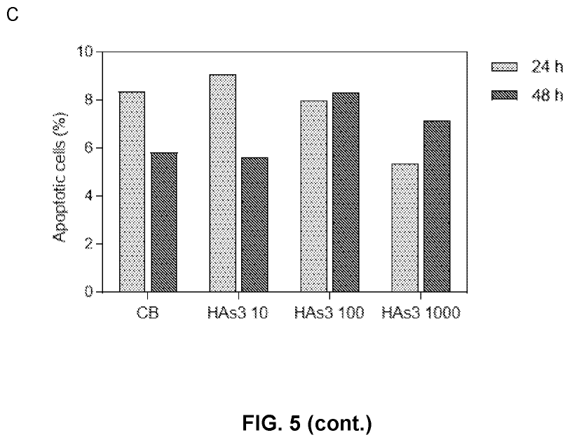

When analyzing the levels of apoptosis found in HUCPVCs treated with HA and HAs, no significant differences were detected between the levels after treatments at the doses used with respect to CB. However, in agreement with the results obtained for cell viability from the MTS assays, a trend towards a reduction in the percentage of apoptotic cells was observed 48 h after initiation of the treatments compared to the 24 h treatments (FIG. 5), although these differences were not statistically significant at any of the concentrations evaluated.

Taken together, these results indicate that the observed reduction in cell viability was related to high doses (1000 μg/ml) of HA and HAs concentration used in the conditioned medium, but not to the chemical composition of these compounds. Moreover, the rest of the concentrations did not affect viability. This confirmed the biological compatibility between HUCPVCs and HA and its sulfated derivatives and allowed defining 48 h as the optimal time to perform the treatments.

### Example 2-Evaluation of Immunogenic Capability of HUCPVCs Treated with HA and HAs

The cellular immune response is the most important mediator of allogeneic transplant rejection and an important barrier to tolerance of this type of transplantation. It is largely mediated by T-cell populations specific for allo-peptides presented in allo-MHC (direct antigen presentation on the major histocompatibility complex) or in self-MHC (indirect antigen presentation on the major histocompatibility complex).

To evaluate the feasibility of using HUCPVCs in cell therapy, it was decided to analyze whether these cells exhibited immunogenicity under physiological conditions and were able to retain their immunosuppressive properties after treatment with HA and HAs, thus being permissive for allogeneic transplantation. For this purpose, a mixed lymphocyte culture (MLC) was performed using HA- and HAs-treated and untreated HUCPVCs and allogeneic lymphocyte proliferation was measured by flow cytometry with the fluorescent dye carboxyfluorescein (CFSE).

### Isolation of Peripheral Blood Lymphocytes by the Ficoll-Paque Plus Gradient

To perform this assay, 60 ml of peripheral blood was initially collected from healthy donors, after signing an informed consent form, in order to isolate peripheral blood mononuclear cells (PBMCs) by Ficoll-Paque Plus gradient (GE Healthcare Bio-Sciences AB, Uppsala, Sweden).

Blood was processed immediately and lymphocytes were purified from PBMCs by the adherence technique. For this purpose, the blood was diluted to half with PBS 1X and, subsequently, Ficoll-Paque was loaded into 50 ml tubes, considering the ratio of one part of Ficoll for every 4 parts of blood diluted to half. Then, the blood diluted to half was seeded on the Ficoll. It was centrifuged to 2000 rpm during 20 minutes, unstopped (ac:6 dc:0). The interphase of the Ficoll gradient was collected by Pasteur pipette, washed 3 times with PBS 1X and centrifuged again at 2000 rpm for 10 minutes (ac:9 dc:9). In the third wash it was centrifuged at 1400 rpm during the same time period. Finally, the supernatant was discarded and the pellet was resuspended in complete RPMI medium without FBS.

PBMCs were placed in a 60 cm² diameter plate and, after 2 h, lymphocytes were recovered from the supernatant, discarding the monocytes adhered to the plate.

### Lymphocytes Stimulation

Isolated lymphocytes were incubated with the PMA/lonomycin mitogen, at a final concentration of 25 ng/ml and 1 μg/ml, respectively, in order to trigger nonspecific lymphocyte activation in vitro and promote their proliferation.

Lymphocyte Labeling with CFSE

After completion of the stimulation with PMA/lonomycin, the lymphocytes were labeled with the fluorescent dye CFSE. For this purpose, lymphocytes were centrifuged at 1400 rpm for 10 minutes and washed with sterile RPMI medium. The pellet obtained was resuspended in PBS-CFSE solution and incubated for 30 min at 37 °C. For the preparation of the PBS-CFSE solution, a volume such that the final concentration was 1×10⁶ cells/ml was considered, and 2 μL of 5 mM/mL stock CFSE solution was added for a final working concentration of 5 UM, as established by the kit manufacturer. Complete RPMI was added (5 times the volume of PBS-CFSE) to stop the staining, the system was incubated on ice for 5 minutes and 3 washes with medium were performed at 1400 rpm for 10 minutes. A fraction of lymphocytes was separated for the control without CFSE labeling. All these steps were carried out in sterility and in darkness.

### Establishment of the MLC

Finally, the MLC was established in which peripheral blood lymphocytes, stimulated and labeled with CFSE, were used as responder cells and cultured with allogeneic stimulatory HUCPVCs previously treated with HA and HAs for 48 h, in a 1:10 ratio (HUCPVCs: lymphocytes). The MLC was performed in complete RPMI medium for 72 h and the necessary controls were included, in addition to different internal controls. Among them, a basal proliferation control with responder lymphocytes without HUCPVCs; a control with CFSE-labeled responder lymphocytes at the time of cytometer reading, such that they do not undergo proliferation rounds (labeling maximum), and an allogeneic control with responder lymphocytes and PBMCs from a second donor. These controls were useful for comparing with the basal proliferation values.

### Measurement of Allogeneic Lymphocyte Proliferation

72 h after initiation of co-culture, allogeneic lymphocyte proliferation was measured by flow cytometry analysis with the fluorescent dye CFSE. CFSE allowed monitoring lymphocyte proliferation due to its progressive and equal distribution within the daughter cells after each cell division. CD3 antigen staining was performed to specifically identify the lymphocyte population of interest. Sample analysis was performed on a FACS Canto II flow cytometer (BD BioSciences) and the data obtained were analyzed using the software FlowJo, LLC.

### Results

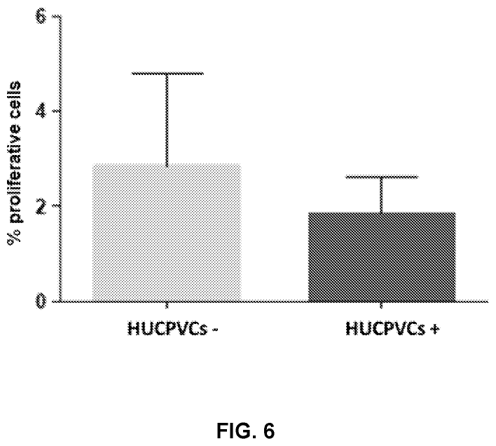

The level of proliferation of responder lymphocytes in the presence (HUCPVCs+) and absence (HUCPVCs−) of the stimulator HUCPVCs was compared. The % of proliferating lymphocytes in the absence of stimulator cells, HUCPVCs−, indicates the level of basal proliferation of responder cells. In this regard, it was observed that HUCPVCs did not trigger an intense lymphocyte reaction in vitro, since no increase in proliferation was observed with respect to the basal proliferation level, which would indicate the low immunogenicity of HUCPVCs. Moreover, a decreasing trend, although not statistically significant, was observed in the proliferation of lymphocytes cultured in the presence of HUCPVCs with respect to their basal proliferation (FIG. 6). These results confirm that HUCPVCs do not exert immunogenic activation on lymphocytes.

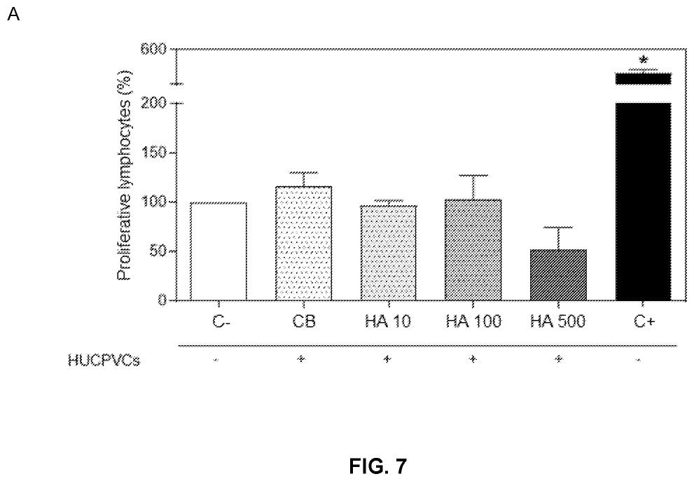

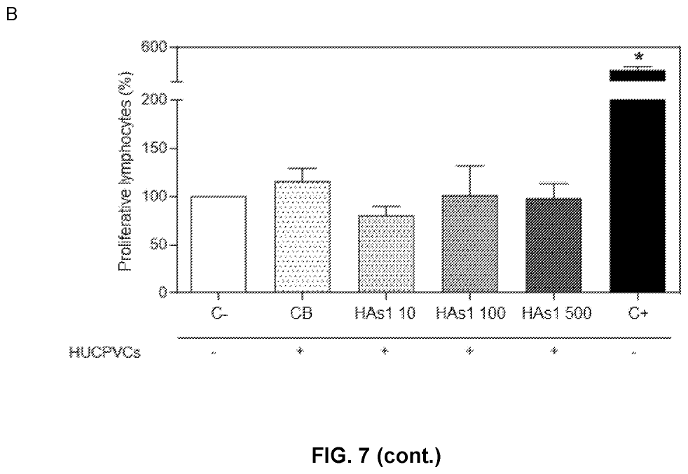

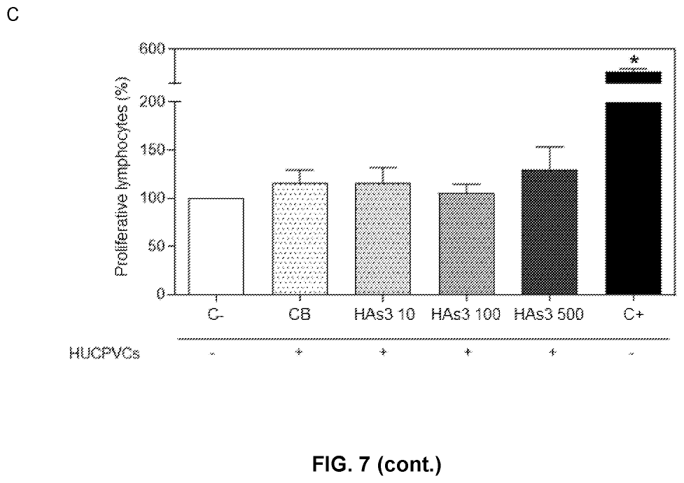

On the other hand, to evaluate whether HA and HAs affect the immune properties of HUCPVCs, the assay was performed on lymphocytes exposed to cells treated with these compounds at the different working concentrations. The results indicate that treatments with HA and its derivatives do not alter the low immunogenicity that characterizes these cells, since no significant differences were observed between lymphocytes exposed to HA and HAs-treated and untreated cells at any of the concentrations evaluated (FIG. 7).

These results indicate that HA and HAs under study do not present a role as in vitro modulators of the immune properties of HUCPVCs, maintaining their immunosuppressive properties, critical for allogeneic cell therapy.

### Example 3-Analysis of Differentiation Capability of HUCPVCs Treated with HA and HAs

To evaluate the contribution of HUCPVCs to bone regenerative capacity and to be able to analyze whether different types of hyaluronic acid affect the in vitro differentiation of these cells to osteogenic and adipogenic lineages, the cells were exposed to the corresponding standard differentiation media, and their degrees of differentiation to each lineage were comparatively analyzed. Cells were either previously exposed or not for 48 h to conditioned medium containing HA or HAs. Then, it was proceeded as indicated below:

Briefly, HUCPVCs were plated in 24-well plates at a density of 5000 cells/cm², where they were maintained for 2 days in standard culture medium until 90% confluence was reached. Upon reaching optimal confluence, the cells were exposed to the differentiation media.

### 1. Osteogenic Differentiation

To induce osteogenic differentiation, once confluence was reached, the standard culture medium was removed and a rinse was performed with 1 ml of warm PBS per well. Next, 500 μL of osteogenic differentiation medium (OM) per well comprising DMEM with low glucose concentration (1 g/L) supplemented with 10% FBS, insulin (10 μg/mL), 50 μg/mL ascorbic acid, 100 nM dexamethasone and 10 mM Naβ-glycerophosphate were added. The medium was changed three times a week, rinsing with warm PBS prior to each change. The differentiation time was 19 days and subsequently the detection of calcification areas was carried out by staining with Alizarin Red S by microscopy.

1.1. Detection of Calcification Areas by Staining with Alizarin Red S

For staining, the differentiation medium was removed and the cells were washed with warm PBS, using 1 ml per well. 3 rinses were performed. The plate was allowed to dry and the cells were fixed with 1 ml of 4% paraformaldehyde (PFA) per well for 2 h. Subsequently, the PFA was removed and 5 rinses were performed with double distilled water. The plate was allowed to dry, 1 ml of 2% Alizarin Red S was added and the plate was left to incubate in the presence of the dye for 10 minutes, under agitation. The dye was removed and 3 rinses were performed with double distilled water to remove the excess. Finally, images were obtained for both qualitative and quantitative analysis using a digital camera (Infinity3-Lumenera) coupled to an inverted brightfield microscope (Zeiss). The technique revealed the areas of calcification resulting from intracellular and extracellular calcium accumulation, showing an intense brown color.

### 1.2. Quantification of Integrated Density of Calcification Zones

For integrated density quantification, the obtained images were analyzed with ImageJ software and expressed in RawIntDen units representing the sum of the values of all pixels in the stained region of interest (ROI) selection. A total of 8 different areas per treatment were analyzed. On the one hand, the integrated density was grouped into ascending intervals of 400 RawIntDen units, which were labeled with the corresponding class label (half limit) in order to obtain a relative frequency distribution of the integrated density of calcification areas. On the other hand, total differentiation towards the osteogenic lineage was quantified as the integrated density in RawIntDen units.

### 1.3. Results

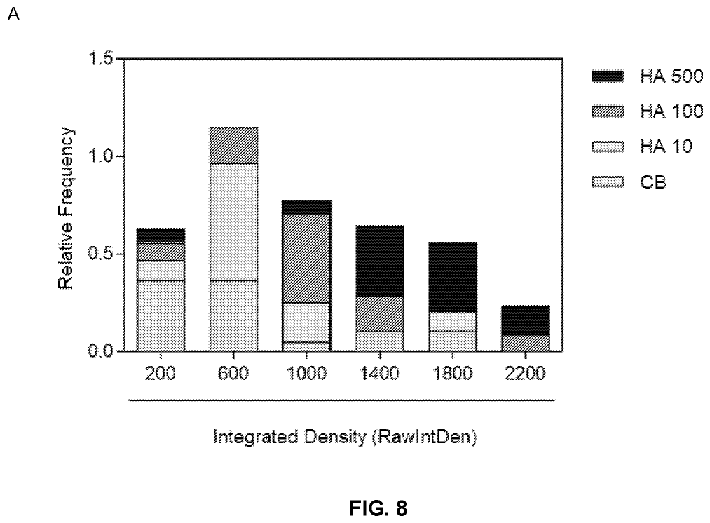

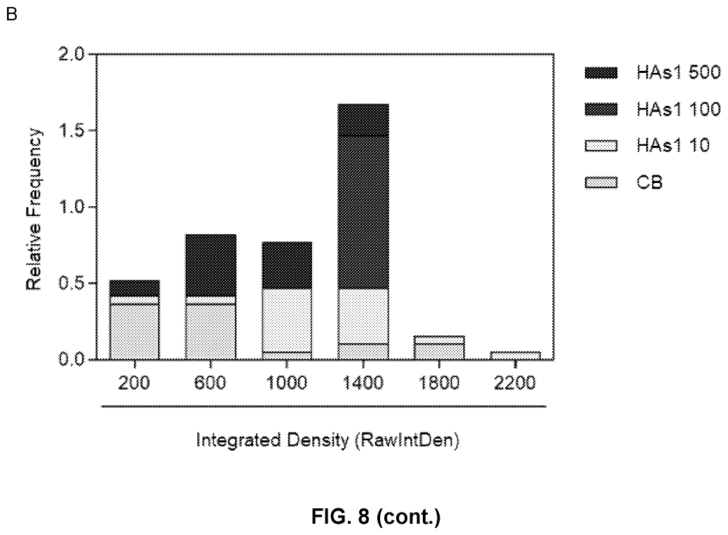

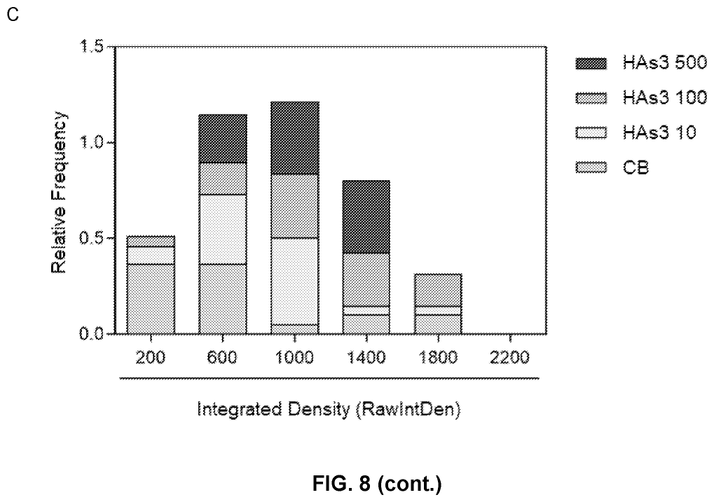

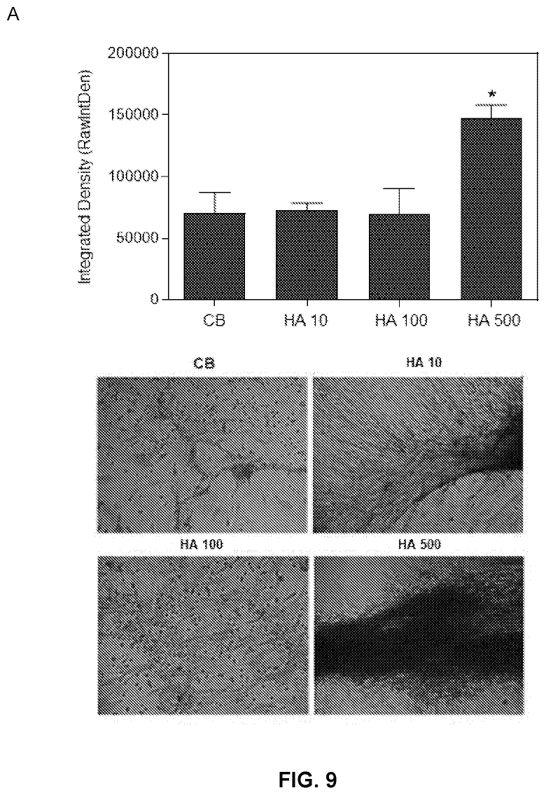

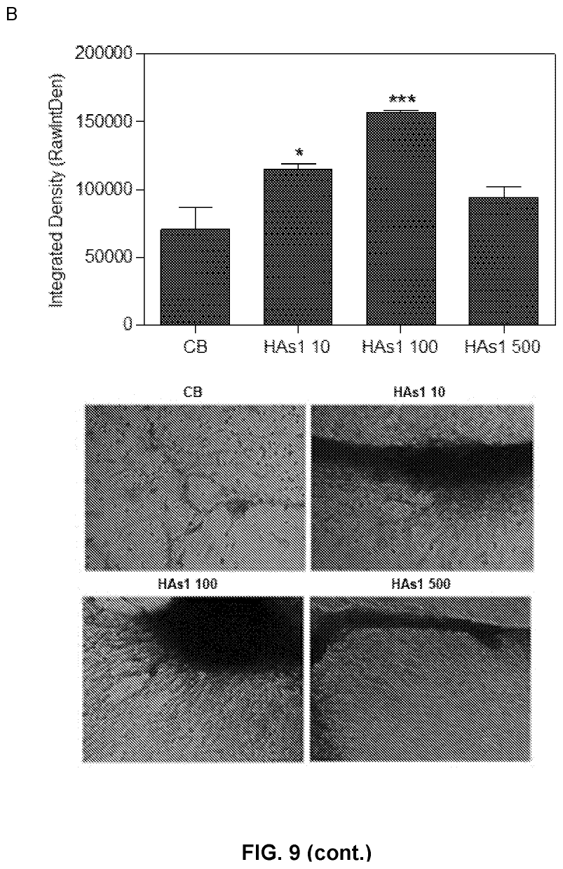

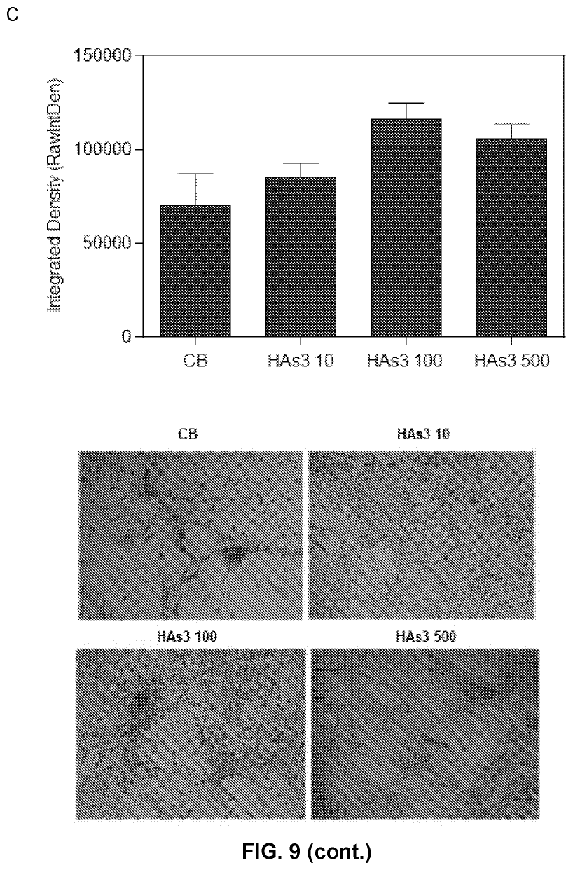

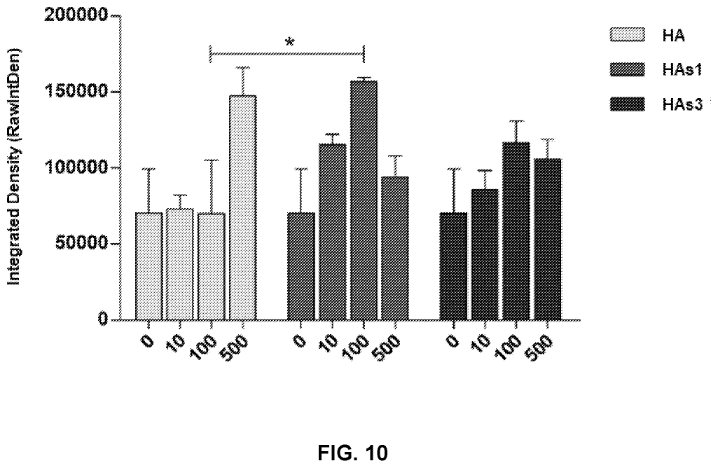

Frequency distribution analysis showed that, in cells previously treated with the conditioned medium comprising HA and HAs, the density of calcification areas during osteogenic differentiation was most frequently contained in the medium-high density intervals (from 801 to 2400 RawIntDen), contrary to untreated cells, in which the density was mostly contained in the medium-low density sections (from 0 to 1600 RawIntDen) (FIG. 8). However, quantification of the integrated density of calcification areas, once differentiation was completed, showed that the ability to differentiate towards the osteogenic lineage of HUCPVCs was preserved after the treatments, since no significant differences were observed with respect to CB. Moreover, differentiation towards this lineage showed a significant increase after HA and HAs1 treatments compared to the control, with no differences observed with HAs3 treatment (FIG. 9). Notably, it was observed that HAs1 had a significantly greater positive effect on osteogenic differentiation with respect to that of HA at the concentration of 100 μg/ml (HA 100=70139+35026; HAs1 100=156843+2550) (FIG. 10).

### 2. Adipogenic Differentiation

To induce adipogenic differentiation, once confluence was reached, the standard culture medium was removed and a rinse was performed with 1 ml of warm PBS per well. 1 ml of adipogenic differentiation medium (MA) was added per well, comprising DMEM with low glucose concentration (1 g/L) supplemented with 10% FBS, 10 μg/mL insulin, 0.5 μM hydrocortisone, 0.5 μM isobutylmethylxanthine (IBMX) and 60 μM indomethacin. The medium was changed three times a week, alternating between the addition of MA in the first change and rest medium in the second change. “Rest medium” refers to DMEM medium with low glucose concentration (1 g/l) and supplemented only with 20% FBS, washing with PBS prior to each change. The differentiation protocol lasted 19 days, and subsequently the detection of lipid droplets was performed by staining with Oil Red O dye by microscopy.

2.1. Detection of Lipid Droplets by Staining with Oil Red O

For staining, the cells were washed with PBS, using 1 ml per well. Subsequently, the cells were fixed with 1 ml of 4% PFA per well for 2 h. The PFA was removed and 5 rinses were performed with double distilled water. Finally, 1 ml of Oil Red O per well was added and the plate was left in mild agitation for 3 h. Three washes were performed with double distilled water, and finally, images were obtained for both qualitative and quantitative analysis using a digital camera (Infinity3-Lumenera) coupled to an inverted brightfield microscope (Zeiss). The technique revealed the presence of lipid accumulations or vacuoles, which were observed to be of an intense red color.

### 2.2. Quantification of Integrated Density of Lipid Vacuoles

For integrated density quantification of lipid vacuoles, the obtained images were analyzed with ImageJ software and expressed in RawIntDen units representing the sum of the values of all pixels in the stained region of interest (ROI) selection. A total of 8 different areas per treatment were analyzed. On the one hand, the integrated density was grouped into ascending intervals of 400 RawIntDen units, which were labeled with the corresponding class label (half limit) in order to obtain a relative frequency distribution of the integrated density of lipid vacuoles. On the other hand, total differentiation towards the adipogenic lineage was quantified as the integrated density in RawIntDen units.

### 2.3. Results

After induction with MA, it was possible to visualize the appearance of lipid vacuoles, which increased in size and number as the incubation time increased in the presence of the medium. The formation of lipid vacuoles indicates differentiation towards this lineage, and was finally confirmed by staining with the dye Oil Red O. The dye revealed the presence of lipid accumulations of an intense red color. Although differences in the number of lipid vacuoles formed at the different concentrations of HA and HAs used could be observed during this follow-up in culture, these were not confirmed until staining with the dye and quantification of the integrated density by ROI.

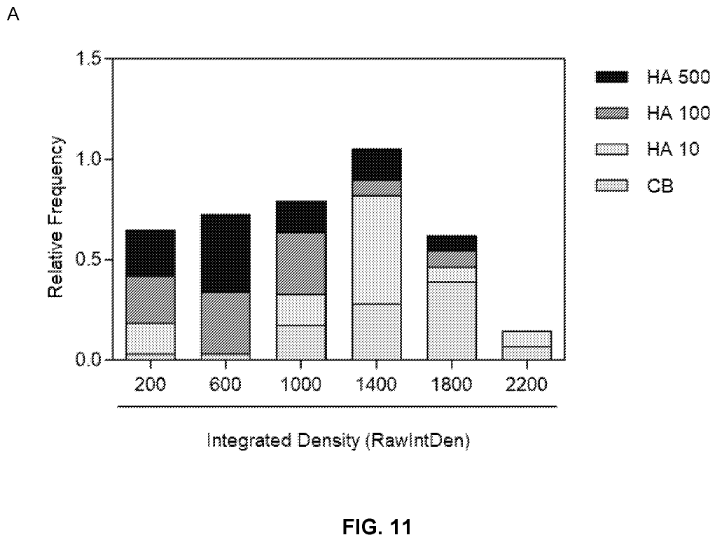

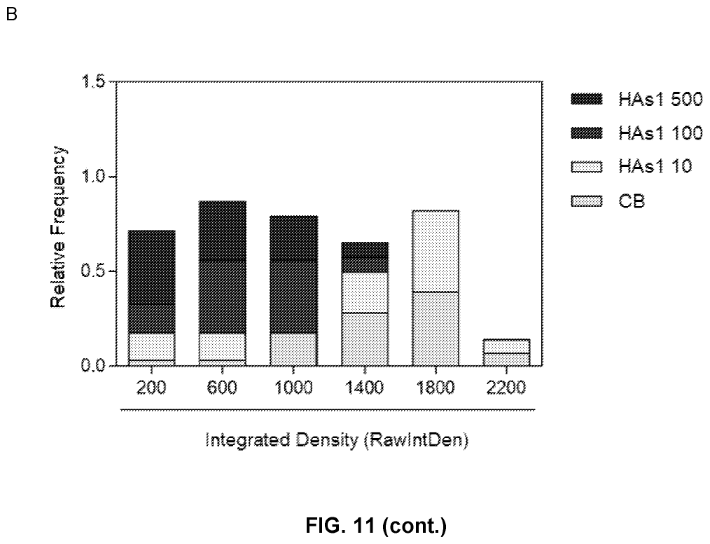

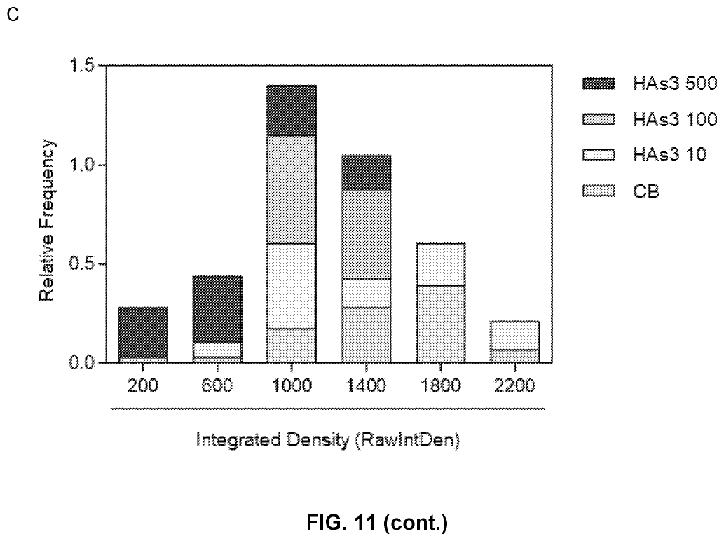

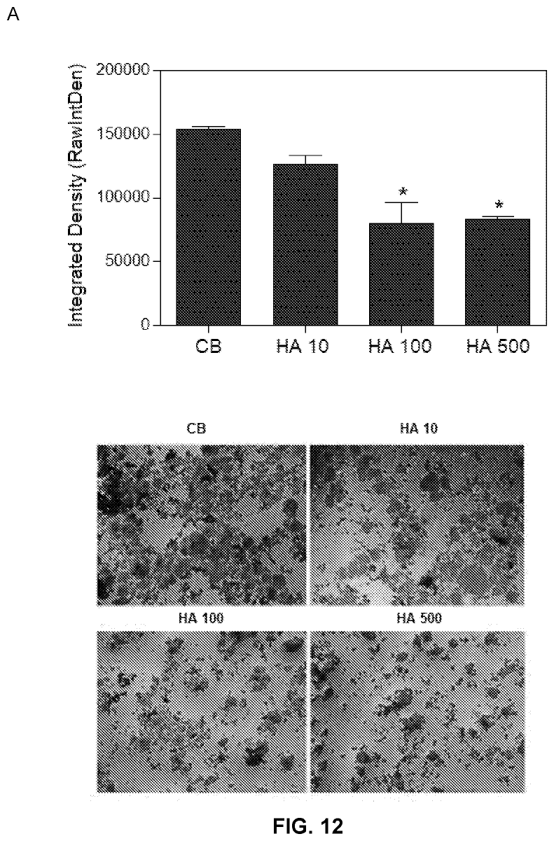

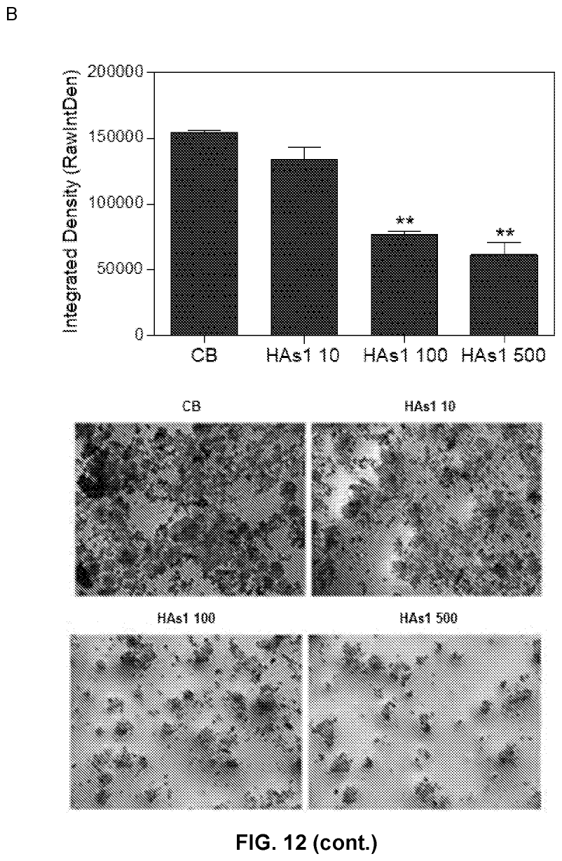

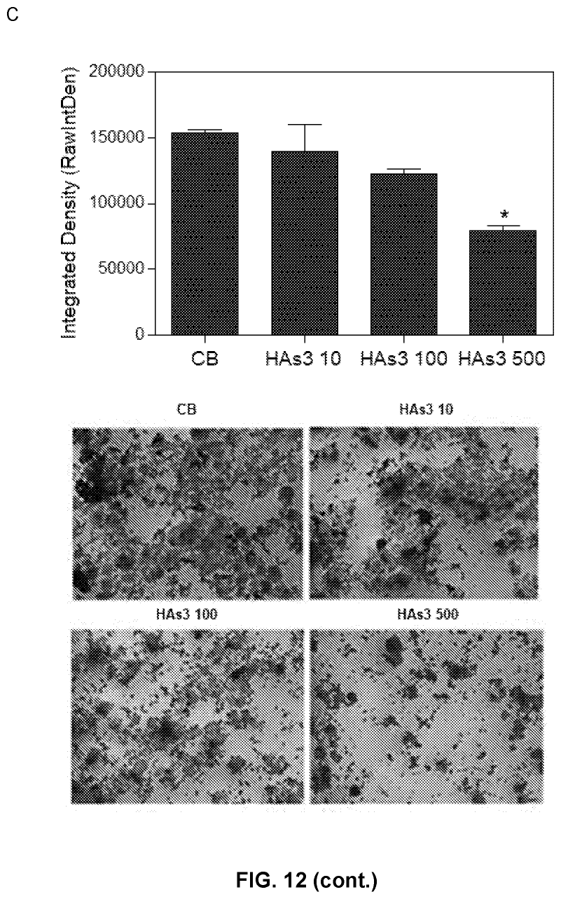

In this sense, the quantification of the integrated density showed that the previous addition of HA and HAS reduced the differentiation towards the adipogenic lineage in vitro, being its effect more marked as the concentration used increases. This was initially evident in the frequency distribution analysis which showed that, in HA and HAs-treated cells, the lipid droplet density obtained for adipogenic differentiation was most frequently contained in the low-medium density intervals (from 0 to 1600 RawIntDen), contrary to untreated cells, in which lipid droplet density was most frequently present in the medium-high density categories (from 801 to 2400 RawIntDen) (FIG. 11). Moreover, when comparing the integrated density of lipid droplets after 19 days of differentiation with respect to CB (154267±2123), it was observed that the treatments with HA (HA 10=126538±6897; HA 100=80100±16892; HA 500=83449±2488) and HAs1 (HAs1 10=134098±8849; HAs1 100=77095±1773; HAs1 500=61345±9364) significantly reduced the differentiation of HUCPVCs towards the adipogenic lineage at the highest concentrations used (500 μg/ml and 1000 μg/ml) (FIGS. 12A and B). However, for HAs3 (HAs3 10=140124±20097; HAs3 100=122605±3615; HAs3 500=79299±3671) a significant reduction was only observed at the highest concentration used (1000 μg/ml) (FIG. 12 C).

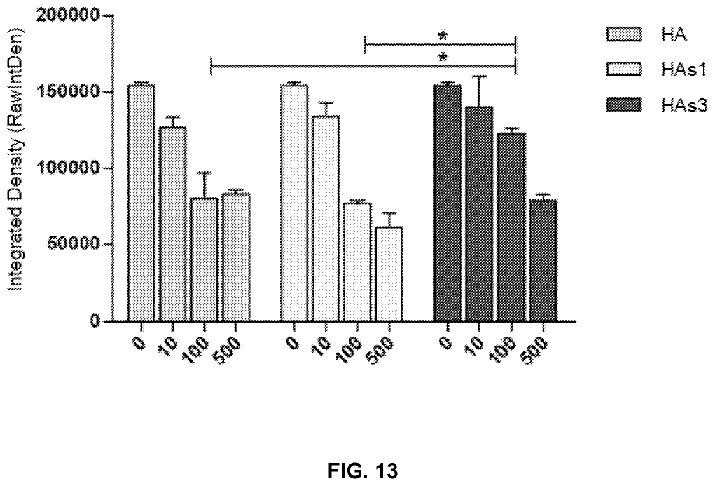

Furthermore, it was observed that the effect of HA and HAs1 was significantly greater with respect to that of HAs3 at the concentration of 100 μg/ml (HA 100=80100+16892; HAs1 100=77095+1773; HAs3 100=122605+3615) (FIG. 13).

### Example 4-Assay in an In Vivo Bone Injury Model

### Fine-Tuning and Development of the In Vivo Bone Injury Model

In order to study the signs of bone regeneration in vivo, the fine-tuning and development of an in vivo model of bone injury was completed and an assay with laboratory animals was performed to show the technical effect of the present invention. This model involved generating a unilateral injury by perforation with an electric drill with a 1 mm diameter bit in the anterior portion of the diaphysis of femurs of 6- to 8-week-old BALB/c animals. The transverse perforations reached the cortical, periosteal and endosteal surface and extended through the bone, without reaching the bone marrow (Me). Once the lesion was generated, MSCs previously treated with HAs1 or HAs3 for 48 h or untreated (as a control group) were locally injected using sterile PBS as a carrier. To ensure animal welfare, during and after generating the injury, an anesthetic and analgesia plan was used in which ketamine (100 mg/kg) and xylazine (10 mg/kg) were combined in the same syringe and administered intraperitoneally. This combination was effective in covering the surgical plane. In addition, to guarantee animal welfare after the bone injury was provoked, and considering that the analgesic effect of xylazine does not last more than 30 minutes, analgesia was supplemented before 30 minutes and for 2 days post-surgery in an effective or preventive manner. Once the analgesia plan was completed, the animals showed no signs of pain. For post-surgical analgesia, ibuprofen (30 mg/kg) was used orally in all cases. In addition, it was verified that the animals did not exhibit peri- or postoperative fractures. In this regard, all animals were able to walk normally 24 h after surgery. Slaughter was performed 28 days post-surgery and femoral samples were obtained. All the protocols used were approved by the regional ethics committee (Committee on Ethics in Research and the Use of Experimental Animals of the National University of the Northwest of the Province of Buenos Aires-COENOBA) ETHICAL EVALUATION OPINION 001/2022. The soft tissue was removed and the femurs were fixed for 24 h in 4% formalin and then embedded in paraffin according to routine procedure, after decalcification with EDTA. For the decalcification step, the bone samples were placed in 0.5 M EDTA (pH=8) for 48 h, performing 6 EDTA replacement cycles of 8 h each. Finally, 5 μm thick longitudinal sections were obtained with microtome and stained with hematoxylin and eosin (H&E) for histopathological analysis. This analysis made it possible to evaluate, under optical microscopy, the signs of bone repair of the induced diaphyseal defect in each treatment condition.

### Experimental Results of the Application of the Invention

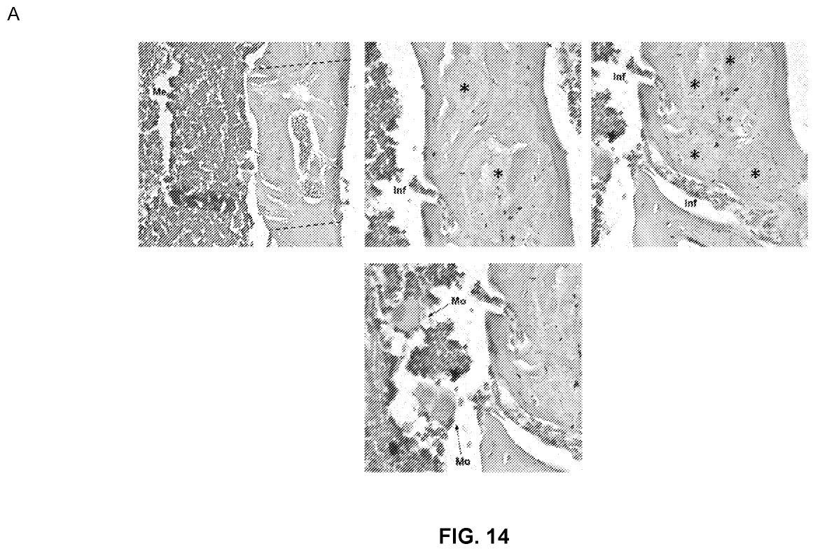

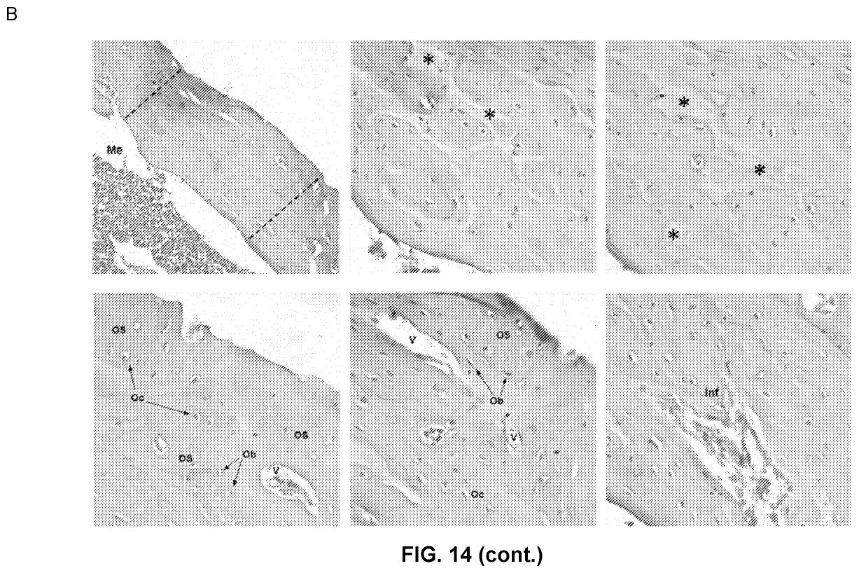

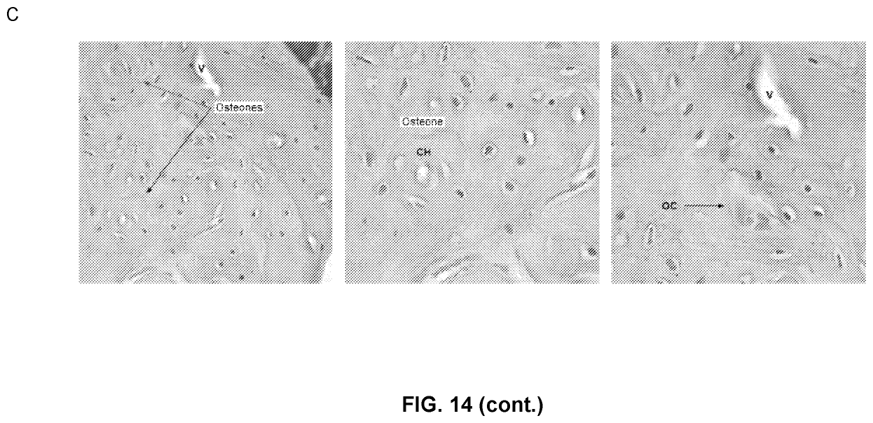

FIG. 14 shows the results obtained 28 days after surgery.

In the control group, treated with HUCPVCs not incubated in the conditioned medium, vacant defects are observed, with absence of osteoid tissue (non-mineralized organic matrix). In addition, areas with strong infiltration of migrant inflammatory cells are found in the injured area that constitute the initial cellular component of the injury environment and presence of mononuclear immune cells near the injury.

In the group treated with HUCPVCs HAs1 tissue is found in different stages of remodeling and maturation. On the one hand, some areas with small vacant defects are observed, being smaller in size than those of the control group. The presence of osteoid tissue was also observed in the area of the injury, this tissue being a non-mineralized organic matrix secreted by osteoblasts during osteogenesis. In addition, the presence of osteoblast-like cells was observed at the periphery of the osteoid tissue, and osteocytes were clearly recognizable within the osteoid tissue due to their morphology. In line with the characteristics of the osteoid tissue, the cells were in randomized position, with apparent presence of unoriented collagen-like fibers. Also, invasion by blood vessels in the area of the bone defect and scarce infiltration of inflammatory cells could be found.

In the group treated with HUCPVCs HAs3, primary osteone-like structures and Havers' canals were observed, which refer to an advanced stage of remodeling and maturation with respect to those previously observed in the other groups. In addition, invasion by blood vessels in the area of the bone defect and presence of multinuclear cells of the osteoclast lineage in the central section could be found. No inflammatory cell infiltration was found in this condition.

In summary, 28 days after surgery, large differences were observed in the bone repair process between the groups evaluated. In principle, the absence of osteoid tissue and the strong presence of immune cells at the site of injury in the control group evidences an initial or immature state of repair. The best regeneration results were observed in the groups in which the lesions were implanted with HUCPVCs treated with sulfated HA derivatives. In this sense, the presence of highly vascular and cellularized tissue, with areas of osteoid tissue and osteoblasts in the periphery, refers to an advanced stage of bone repair in the group treated with HUCPVCs HAs1. Even more so, in the group treated with HAs3, where the presence of osteonal structures (osteones) and delimitation of haversian canals was observed, indicative of a more mature remodeling and in the final stage of the bone regeneration process.

### Example 5-Analysis of Antitumoral Capability of the Culture Supernatants of HUCPVCs Treated with HA and HAs

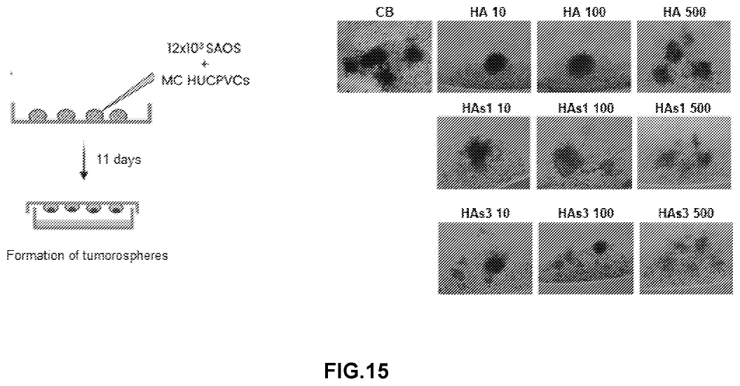

To date, no data are available on the interaction between MSCs and HA and HAs on the tumor context. FIG. 15 shows the results obtained in vitro of the effect of the culture supernatants of HA and HAs-treated and untreated HUCPVCs on the viability and tumor behavior of the osteosarcoma (OS) cell line SAOS-2 in order to analyze the effect of the soluble factors secreted by this cell product under the different conditions. Tumor cell behavior was assessed by 3D assays of in vitro tumorosphere formation. To perform the 3D culture of tumorospheres, 4 drops per treatment were prepared with 12×10³ SAOS-2 cells per drop in a final volume of 20 μl. Supernatants derived from HUCPVCs treated with HA and HAs and untreated for 48 h were used as treatment. The droplets were placed on the lid of a 60 cm² diameter plate, using PBS on the bottom surface. Cells were incubated for 11 days and culture growth was monitored. It was observed that the supernatant derived from this product produced a decrease in the number of tumorospheres formed in 3D and a greater formation of cell aggregates, an effect that was increasingly greater as the concentration of HA and HAS increased for the range of concentrations evaluated. This effect is indicative that soluble factors derived from HUCPVCs treated with HA and HAs appear to reduce the initial intercellular adhesion conducive to the formation of new stable tumor masses, limiting the tumorigenic capacity of OS cells.

### REFERENCES

Hench L. L., Polak J. M. Third-generation biomedical materials. Science. 2002; 295:1014-1017. doi: 10.1126/science. 1067404
Hintze V, Moeller S, Schnabelrauch M, et al. Modifications of hyaluronan influence the interaction with human bone morphogenetic protein-4 (hBMP-4). Biomacromolecules. 2009; 10 (12): 3290-3297. doi: 10.1021/bm9008827.
Iaquinta M R, Mazzoni E, Bononi I, et al. Adult Stem Cells for Bone Regeneration and Repair. Front Cell Biol. 2019; 7:268. Published 2019 Dev November 12. doi: 10.3389/fcell.2019.00268
Mebarki M, Abadie C, Larghero J, Cras A. Human umbilical cord-derived mesenchymal stem/stromal cells: a promising candidate for the development of advanced therapy medicinal products. Stem Cell Res Ther. 2021; 12 (1): 152. Published 2021 Feb. 26. doi: 10.1186/s13287-021-02222-y
Meijer G J, de Bruijn J D, Koole R, van Blitterswijk C A. Cell based bone tissue engineering in jaw defects. Biomaterials. 2008; 29 (21): 3053-3061. doi: 10.1016/j.biomaterials.2008.03.012
Pavan M, Beninatto R, Galesso D, et al. A new potential spreading factor: Streptomyces koganeiensis hyaluronidase. A comparative study with bovine testes hyaluronidase and recombinant human hyaluronidase of the HA degradation in ECM. Biochim Biophys Acta. 2016; 1860 (4): 661-668. doi: 10.1016/j.bbagen.2015.12.024

1. A method for promoting osteogenic differentiation of mesenchymal stem cells (MSCs), characterized by comprising:
i—providing a standard culture medium suitable for the incubation of MSCs on a plate;
ii—seeding MSCs in the standard culture medium;
iii—incubating the MSCs in the standard culture medium to achieve their cell amplification;
iv—adding hyaluronic acid or a sulfated derivative thereof to the standard culture medium to generate a conditioned medium; and
v—incubating the MSCs in said conditioned medium.

2. The method of claim 1, characterized in that the MSCs are obtained from human umbilical cord tissue (hUC-MSCs).

3. (canceled)

4. The method of claim 1, characterized in that step iv—consists of adding a sulfated derivative of hyaluronic acid having an average amount of sulfate groups per repeating unit of disaccharide within the range between 1 and 3.

5. The method of claim 4, characterized in that the sulfated derivative of hyaluronic acid has an average amount of sulfate groups per repeating unit of disaccharide selected from the group consisting of 1 and 3.

6. (canceled)

7. The method of claim 1, characterized in that the hyaluronic acid or the sulfated derivative thereof is added to the standard culture medium in a concentration of between 10 μg/ml and 1000 μg/ml.

8. (canceled)

9. The method of claim 1, characterized in that the MSCs are incubated in the conditioned medium for 48 h.

10. The method of claim 1, characterized in that, in step ii—, the MSCs are seeded in a plating density in the range between 5×10³ and 7.5×10³ cells/cm².

11. (canceled)

12. A composition for use in bone regenerative therapy characterized by comprising MSCs obtained from the method for promoting osteogenic differentiation of MSCs according to claim 1, suspended in a pharmaceutically acceptable carrier.

13. The composition of claim 12, characterized in that the MSCs comprised within the composition are hUC-MSCs.

14. (canceled)

15. The composition of claim 12, characterized in that it comprises MSCs induced towards osteogenic differentiation by their incubation with a sulfated derivative of hyaluronic acid which has an average amount of sulfate groups per repeating unit of disaccharide within the range between 1 and 3.

16. The composition of claim 15, characterized in that the sulfated derivative of hyaluronic acid has an average amount of sulfate groups per repeating unit of disaccharide selected from the group consisting of 1 and 3.

17. (canceled)

18. A method for the regenerative treatment of a bone injury in a subject, characterized by comprising:
i—providing a standard culture medium suitable for the incubation of MSCs on a plate;
ii—seeding MSCs in the standard culture medium;
iii—incubating the MSCs in the standard culture medium to achieve their cell amplification;
iv—adding hyaluronic acid or a sulfated derivative thereof to the standard culture medium to generate a conditioned medium;
v—incubating the MSCs in said conditioned medium; and
vi—inoculating the MSCs obtained after the incubation of step v—in the bone injury.

19. The method of claim 18, characterized in that the MSCs are hUC-MSCs.

20. (canceled)

21. The method of claim 18, characterized in that step iv—consists of adding a sulfated derivative of hyaluronic acid having an average amount of sulfate groups per repeating unit of disaccharide within the range between 1 and 3.

22. The method of claim 21, characterized in that the sulfated derivative of hyaluronic acid has an average amount of sulfate groups per repeating unit of disaccharide selected from the group consisting of 1 and 3.

23. (canceled)

24. The method of claim 18, characterized in that the hyaluronic acid or the sulfated derivative thereof is added to the standard culture medium in a concentration of between 10 μg/ml and 1000 μg/ml.

25. (canceled)

26. The method of claim 18, characterized in that the MSCs are incubated in the conditioned medium for 48 h.

27. The method of claim 18, characterized in that, in step ii—, the MSCs are seeded in a plating density in the range between 5×10³ and 7.5×10³ cells/cm².

28. (canceled)

29. The method of claim 18, characterized in that the inoculation of the MSCs is carried out locally in the site of the bone injury.

30. The method of claim 18, characterized in that the bone injury is caused by a tumor.
